(19) United States
(12) Patent Application Publication
(10) Pub. No.: US 2004/0042959 A1
(43) Pub. Date: Mar. 4, 2004

(54) Imaging cell death in vivo using non-radionuclide contrast agents

(76) Inventors: Michael Montalto, Loudonville, NY (US); Bruce Johnson, Scotia, NY (US); Mohan Amaratunga, Clifton Park, NY (US)

Correspondence Address: Tracy R. Loughlin, Dougherty, Clements & Hofer, 1901 Roxborough Road, Suite 300, Charlotte, NC, 28211, US

(21) Appl. No.: 10/229,689

(22) Filed: Aug. 28, 2002

(51) Int. Cl. A61K 51/00; A61K 49/00

(52) U.S. Cl. 424/1.11; 424/9.36; 424/9.6

(57) ABSTRACT

An annexin, annexin analogue or phosphatidylserine binding compound (PSC) labeled with an MR, CT, or optical contrast agent. The conjugate is administered into a subject and specifically binds to the surface of apoptotic and necrotic cells. The subject is imaged using conventional MRI, CT and optical imaging techniques and dead and dying tissue is identified. The identification and development of analogues specific for phosphatidylserine for purposes of non-invasive imaging of dead or dying cells.

### BACKGROUND OF THE INVENTION

1. Field of the Invention

The present invention relates generally to the field of imaging. More specifically, the present invention relates to non-invasive imaging of cell death in vivo using non-radionuclide contrast agents.

2. Description of the Related Art

Non-invasive imaging of cell death in vivo plays an important role in the future of modern medicine. Thus far, only radionuclide based imaging modalities have been extensively explored as potential avenues for imaging cell death in vivo. This technology involves the labeling of the phosphatidylserine-binding-protein, annexin-V, with a radionuclide and subsequent imaging by positron emission tomography (PET) or single photon emission tomography (SPECT). Such radionuclide-dependent scanning modalities provide poor special resolution and are not widely available at most medical facilities. Conversely, magnetic resonance imaging (MRI) and computed tomography (CT) based imaging modalities are widely available, yield outstanding spacial resolution and are minimally toxic. One disadvantage to these modalities, however, is that they are non-specific. Another disadvantage to these modalities is that when using contrast agents, the agents must be present in very high concentrations (milli or micromolar) to achieve adequate levels of detectability.

Magnetic resonance imaging is a technique that uses a powerful magnetic field and radio signals to create sophisticated vertical, cross-sectional, and three-dimensional images of structures and organs inside a body. Unlike conventional radiography, which makes use of potentially harmful radiation (X-rays), MRI imaging is based on the magnetic properties of atoms. MRI is most effective at providing images of tissues and organs that contain water, such as the brain, internal organs, glands, blood vessels, and joints. When focused radio wave pulses are broadcast towards aligned hydrogen atoms in a tissue of interest, the hydrogen atoms return a signal. The subtle differences in the signal from various body tissues enable MRI to differentiate organs, and potentially contrast abnormal tissue. MRI is useful for detecting tumors, bleeding, aneurysms, lesions, blockage, infection, joint injuries, etc.

Cell death plays a pathological role in many disease states including myocardial infarction, transplantation rejection, acute and chronic inflammation, ischemic heart disease, and stroke. Additionally, tracking the extent of cell death during chemotherapeutic regimens provides important information regarding the effectiveness of therapy. Therefore, monitoring cell death in vivo has been the subject of much investigation.

Cell death may result from a number of factors, such as apoptosis, necrosis, lysis, and senescence. Apoptosis is a pre-programmed mechanism in which a cell self-destructs when stimulated by an appropriate trigger. Apoptosis may be initiated when a cell is no longer needed, when a cell becomes a threat to an organism's health, or for other reasons. The aberrant inhibition or initiation of apoptosis contributes to many disease processes, including cancer. Apoptosis is distinguished from necrosis, a form of cell death that results from injury and disease, especially in a localized area of the body. Lysis is the rupturing of the cell membrane and the loss of the cytoplasm. Senescence is the inability to replicate while still maintaining a degree of viability.

All eukaryotic cells are surrounded by an intact plasma membrane that is comprised of a phospholipid bilayer. The extracellular layer is comprised primarily of phosphatidylcholine and sphingomyelin. The inner cytosolic leaflet comprises phosphatidylethanolamine and the negatively charged phosphatidylserine (PS). During cell death, PS may be translocated from the inner membrane to the outer extracellular surface, or may be exposed by cell lysis. Interestingly, the class of proteins known as annexins bind specifically to PS with a high affinity. For example, annexin-V and annexin-V analogues labeled with a chromogen or radionuclide have been used to identify apoptotic cells both in vitro and in vivo. Although labeling annexin-V with radionuclides is an important first step in imaging cell death, there are clear limitations to using radionuclide-dependent sensors. For example, the short half-life of commonly used radionuclides, such as Fluorine 18, limits the window of opportunity to image certain targets. Further, some radionuclide-dependent imaging modalities, such as positron emission tomography (PET), are not readily available to the public. Although using other nuclide-based modalities, such as single photon emission computed tomography (SPECT), may circumvent these limitations, SPECT is not widely accepted in the medical community and yields lower sensitivity compared to PET. Lastly, radionuclide imaging techniques of any sort do not produce the desired spatial resolution required to accurately localize areas of cell death or tissue damage.

A significant limitation in the development of non-radionuclide targeted contrast agents is the lack of sensitivity. It is generally accepted that non-radionuclide imaging modalities such as magnetic resonance (MR) requires a ten to one-thousand fold increase of contrast agent compared to nuclide based modalities. Fortunately, PS translocation during cell death exposes nearly 10 million binding sites for annexins, theoretically approaching the required amount of target needed for MRI imaging. The ability to label annexins with a magnetically active contrast agent, such as gadolinium (Gd) or iron nanoparticles, would provide the resolution that is lacking with PET and SPECT imaging, while maintaining the ability to specifically report on cell death/tissue damage.

Conventional clinical evaluations of cell death have been performed using in vitro serum tests for the enzyme creatine kinase, which is released into the blood by necrotic tissue. This test, however, is crude and does not evaluate the specific location of cell death. Cardiac echocardiograms, thallium uptake and other similar tests that evaluate myocardial function may localize damaged tissue, but do not provide adequate resolution and do not differentiate cell death from cell dysfunction. One standard clinical method to identify viable myocardium is PET, however, metabolic imaging using 18F-fluoro-deoxyglucose (FDG) with standard PET techniques is not adequate for identifying small locally dead regions within so-called “hibernating” tissue.

In cases involving transplant rejection, needle biopsies may be performed to assess rejection, yet non-invasive tests are not currently available. Thus far, the only imaging of cell death in vivo has been experimental and has only been described using radionuclide-labeled annexins, such as annexin-V. Although these studies are the first to show in vivo images of cell death, they are not sufficient to provide the required resolution necessary under certain clinical situations. In addition, they are only available at a limited number of medical facilities that have PET imaging equipment.

What is needed to greatly improve imaging techniques is to create a non-radionuclide MRI based contrast agent that is conjugated to a molecule that can specifically bind to dead or dying cells, such as annexin-V or PS binding compounds (PSCs) that are conjugated to Gd. Further, what is needed is the exploration of alternative imaging modalities, such as optical or computed tomography (CT) based imaging modalities. Still further, what is needed is to address the limitation of having to use annexin-V at low doses by developing analogues that are not biologically active at high doses.

### SUMMARY OF THE INVENTION

In one aspect, the contrast agent of the present invention aids in the detection of physiological changes associated with tissue abnormality, such as cardiovascular disease, thrombosis, cancer, etc. The contrast agent comprised of an annexin complex conjugated to a non-radionuclide contrast agent of nanoparticles is able to selectively attach to dead and dying cells of a subject. The dead and dying cells are important indicators of abnormality and disease.

In another aspect, the present invention provides a method whereby PS binding compounds (PSCs) and annexins, such as annexin-V and annexin-V analogues, are labeled with an MR, CT or optical contrast agent. Annexin-V and PSCs conjugated to the contrast agent are injected into a body and specifically bind to the surface of necrotic and apoptotic cells. After clearance of the non-bound contrast agent, the patient is imaged by an imaging technique, such as MRI, CT or optical techniques, and dying and dead tissues are identified.

In a further aspect, the non-radionuclide contrast agent comprises an MR, CT, or optical contrast agent such as chromium(III), manganese(II), iron(III), iron(II), cobalt(II), nickel(II), copper(II), praseodymium(III), neodymium(III), samarium(III) and ytterbium(III). Because of their very strong magnetic moments, gadolinium (III), terbium(III), dysoprosium(III), holmium(III), and erbium(III) are preferred. Especially preferred for the paramagnetic atom is gadolinium(III). Most preferred are gadolinium or iron containing compounds.

In a still further aspect, the labeled annexin has a high affinity for binding with phosphatidylserine (PS), which is released or exposed by a dead or dying cell during apoptosis and necrosis. PS is translocated from an inner cell membrane to an outer extracellular surface.

In a still further aspect, the present invention comprises the identification and development of PSCs that may include peptides, small molecules, aptomers and antibodies specific for PS or other targets specific to dead and dying cells.

In a still further aspect, the present invention addresses the limitation of having to use annexin-V at a low dose level by developing PSCs that are not biologically active at high dose levels. The PSCs retain at least a native or better affinity for PS.

In a still further aspect, the annexin comprises annexin-V or a derivative of annexin-V having a high affinity for phosphatidylserine.

Another important feature of this invention is the coupling of repeating chelate-Gd or other magnetically active contrast agents so as to provide amplification of a signal. An envisioned embodiment of such an amplification scheme is the creation and use of a genetically or chemically modified annexin or PSCs that contain 1-50 lysines. Each amine residue on lysine may be chemically modified to contain a chelate (DTPA or DOTA) which allows for the binding of Gd or other magnetic metals. Although lysine is the residue of choice, it is possible that other repeating amino acid residues that may be chemically modified to chelate metals may be used.

### BRIEF DESCRIPTION OF THE DRAWINGS

These and other features, aspects, and advantages of the non-invasive imaging of the present invention are better understood when the following Detailed Description of the Invention is read with reference to the accompanying drawings, wherein:

FIG. 1 is a functional block diagram of an MRI system that employs a non-radionuclide contrast agent conjugated to an annexin molecule in accordance with an exemplary embodiment of the present invention.

### DETAILED DESCRIPTION OF THE INVENTION

As required, detailed embodiments of the present invention are disclosed herein, however, it is to be understood that the disclosed embodiments are merely exemplary of the invention that may be embodied in various and alternative forms. Specific structural and functional details disclosed herein are not to be interpreted as limiting, but merely as a basis for the claims as a representative basis for teaching one skilled in the art to variously employ the present invention.

One object of the present invention is to provide contrast agents that aid in the imaging of cells undergoing death. In one embodiment, the present invention comprises labeling phosphatidylserine (PS) binding compounds (PSCs) and annexins, such as annexin-V or annexin-V analogues, with a non-radionuclide contrast agent. The contrast agent comprises an MR, CT or optical contrast agent. Annexins conjugated to the contrast agents are injected into a body and specifically bind to the surface of necrotic and apoptotic cells. A patient may then be imaged using MRI, CT or optical techniques, and images of dead or dying tissue are obtained. One advantageous property of the present invention is the high resolution provided by non-radionuclide MR and CT contrast agents, another is the high availability of MRI and CT scanners at medical facilities.

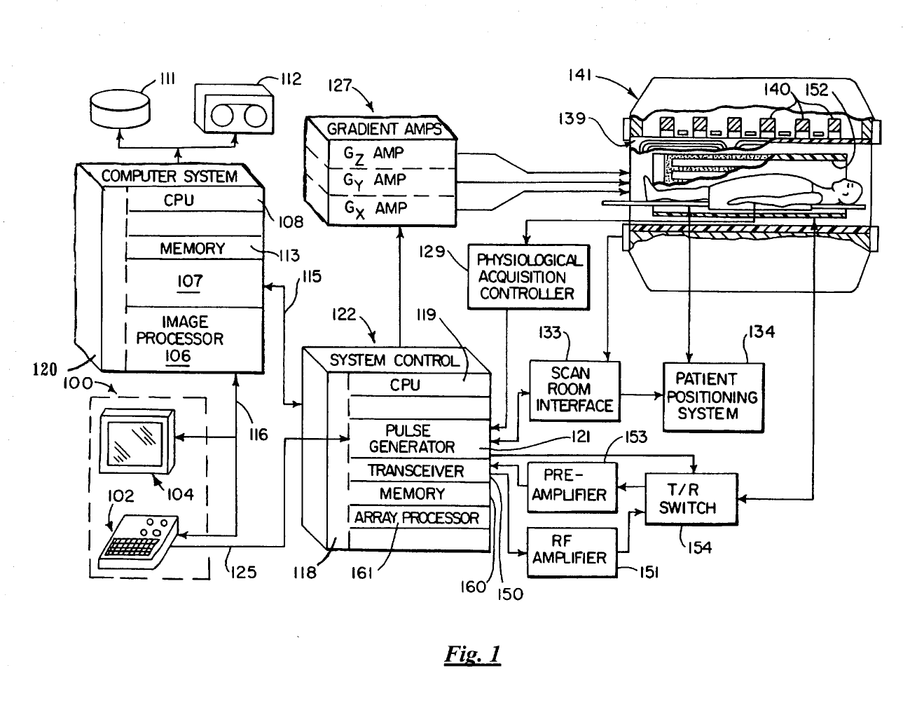

Referring to FIG. 1, an exemplary imaging modality is illustrated that may be used in conjunction with the non-radionuclide contrast agent labeled annexines of the present invention. The operation of the system is controlled from an operator console 100. The console 100 comprises a control panel 102, operable for controlling the system, and a display 104, operable for displaying images. Console 100 communicates with computer system 107 via link 116. Computer system 107 comprises a plurality of modules that communicate via a backplane 120. The plurality of modules comprise image processor module 106, central processing unit (CPU) module 108, and memory module 113, operable for storing image data arrays and known in the art as a frame buffer. Computer system 107 is linked to disk storage 111, tape drive 112, and separate system control 122 via high-speed serial link 115.

System control 122 comprises a set of modules linked together via backplane 118. The set of modules comprise CPU module 119 and pulse generator module 121, which is coupled to operator console 100 via serial link 125. System control 122 receives commands from the operator determining the appropriate scan sequence to administer.

Pulse generator module 121 instructs the system components to carry out the appropriate scan sequence and produces timing data, strength data, the shape of RF pulses to be produced, and the timing and length of the data acquisition window. Pulse generator module 121 is operatively connected to gradient amplifiers 127, which control the timing and shape of the gradient pulses to be produced during the scan. In addition, pulse generator module 121 receives patient data from physiological acquisition controller 129. Signals are transmitted to controller 129 via a plurality of sensors attached to the patient, such as electrocardiogram (ECG) and respiratory signals. Pulse generator module 121 is also operatively connected to scan room interface circuit 133, which receives signals from sensors associated with patient condition and the magnet system. Patient positioning system 134 receives adjustment commands via scan room interface 133.

Gradient amplifier system 127 is comprised of G_{X}, G_{Y} and G_{Z} amplifiers, and receives gradient waveforms from pulse generator module 121. Each gradient amplifier instructs a corresponding gradient coil in assembly 139 to produce the magnetic field gradients used for position encoding acquired signals. Gradient coil assembly 139 forms part of magnet assembly 141, which includes polarizing magnet 140 and whole-body RF coil 152. Transceiver module 150 produces pulses that are amplified by RF amplifier 151 and coupled to RF coil 152 by transmit/receive switch 154. The resulting signals radiated by the excited nuclei in the patient may be sensed by RF coil 152 and coupled to preamplifier 153 via transmit/receive switch 154. The amplified NMR signals are demodulated, filtered, and digitized in the receiver portion of transceiver 150. Transmit/receive switch 154 is controlled by a signal from pulse generator module 121. Switch 154 electrically connects RF amplifier 151 to coil 152 during the transmit mode, and connects preamplifier 153 to coil 152 during the receive mode. Transmit/receive switch 154 also enables a separate RF coil (for example, a head coil or surface coil) to be used in either transmit or receive mode.

The NMR signals received by RF coil 152 are digitized by transceiver module 150 and transferred to memory module 160 in system control 122. When a scan is completed and data is acquired in memory module 160, array processor 161 transforms the data into an array of image data. Image data is conveyed via serial link 115 to computer system 107 and stored in disk storage 111. In response to commands received from operator console 100, image data may be archived on tape drive 112, or may be further processed by image processor 106 and conveyed to operator console 100 for presentation on display 104.

Although the invention may be used with a number of different pulse sequences, an exemplary embodiment of the invention employs a fast 3D (three-dimensional) RF (radio frequency) phase spoiled gradient recalled echo pulse sequence.

A mechanism employed in MRI to provide contrast in reconstructed images is the T₁ relaxation time of nuclear spins in a tissue. After excitation, a period of time is required for longitudinal magnetization to fully recover. This period, referred to as the T₁ relaxation time, varies in length depending on the particular spin species being imaged. Spin magnetizations with shorter T₁ relaxation times appear brighter in MR images acquired using fast T₁ weighted NMR measurement cycles. The level of signal brightness, i.e. signal enhancement, in T₁ weighted images is proportional to the concentration of contrast agents in the tissue being observed.

While an exemplary MRI system has been described above, it is understood that alternative imaging systems may be used, such as computed tomographic (CT) imaging. Tomography is a method of body imaging in which an X-ray source and/or detection device (e.g., film) rotate around a patient. In CT, a thin X-ray beam rotates as small detectors measure the amount of X-rays that pass through the patient or particular area of interest. Using a complex algorithm, a computer analyzes data to construct a cross-sectional (axial) image. The images may be stored, viewed on a monitor or printed on film. In addition, stacking the individual images, referred to as “slices”, creates three-dimensional models of organs.

The contrast agent labeled annexins of the present invention respond to physiological parameters in various ways. In one aspect, the contrast agent responds to physiological parameters associated with cell death by accumulating in an area of an increased specific parameter value, such as phosphatidylserine (PS), as discussed below. Binding causes the contrast agent to be slowed at a site of interest, increasing the resident time. The site of interest, e.g. abnormal tissue, possess specific physiological parameter differences compared to the surrounding tissues. The physiological differences identified and marked by the contrast agent of the present invention lead to an increase in the signal intensity compared to conventional contrast agents. Thus, the contrast agents of the present invention provide a clinician with an improved capability to detect early disease states.

Cell death may be caused by apoptosis, necrosis, senescence, and lysis. These causes of cell death result in cells losing their plasma membrane integrity. Cell death refers to the death or imminent death of nucleated cells as well as to the death or imminent death of anucleate cells (e.g., red blood cells, platelets, etc.). As stated above, apoptosis refers to pre-programmed cell death whereby a cell executes a suicide program. The program is observed among virtually all multicellular organisms, as well as among all the cells in a particular organism. It is also believed that apoptosis may be a default program that must be actively inhibited in healthy surviving cells. Cells undergoing cell death comprise myocytes, hepatocytes, epithelial cells, and cells derived from specific organs such as the liver, kidney, prostate, breast, intestine, bone, and muscle.

The pre-programmed cell decision may be influenced by a variety of regulatory, physiological activators and environmental factors. Physiological activators comprise tumor necrosis factor (TNF), Fas ligand, transforming growth factor A, neurotransmitters glutamate, dopamine, N-methyl-D-aspartate, withdrawal of growth factors, loss of matrix attachment, calcium, and glucocorticoids. Damage-related inducers of apoptosis comprise heat shock, viral infection, bacterial toxins, the oncogenes myc, rel and E1A, tumor suppressor p53, cytolytic T-cells, oxidants, free radicals, and nutrient deprivation (antimetabolites). Therapy-associated apoptosis inducers comprise gamma radiation, UV radiation, and a variety of chemotherapeutic drugs, including cisplatin, doxorubicin, bleomycin, cytosine arabinoside, nitrogen mustard, methotrexate, and vincristine. Toxin-related inducers of apoptosis comprise ethanol and β-amyloid peptide.

Apoptosis has particularly devastating consequences when it occurs pathologically in cells that do not normally regenerate, such as neurons. Cells that are not replaced when they die may lead to debilitating and sometimes fatal dysfunction of the affected organ. Such dysfunction may be evidenced in a number of neurodegenerative disorders associated with increased apoptosis, including Alzheimer's disease, Parkinson's disease, amyotrophic lateral sclerosis, retinitis pigmentosa, and cerebellar degeneration.

Necrosis is the localized death of cells or tissue due to causes other than apoptosis (i.e., other than the execution of the cell's intrinsic suicide program). Necrosis may be caused by injury, infection and the like. There is some overlap between the two types of cell death, in that some stimuli may cause necrosis, apoptosis or both, depending on the severity of the injury.

All eukaryotic cells are surrounded by an intact plasma membrane that is comprised of a phospholipid bilayer. The extracellular layer is comprised primarily of phosphatidyl choline and sphingomyelin. The inner cytosolic leaflet comprises phosphatidylethanolamine and the negatively charged phosphatidylserine (PS). In one example, tumor vasculature comprises PS located in the lumen of the vasculature. During cell death, such as in apoptosis, PS is translocated from the inner membrane to the outer extracellular surface, or may be exposed by cell lysis.

PS exposure is a component in both apoptosis and necrosis. When an apoptotic cell has reached the terminal stage of apoptosis (i.e., loss of membrane integrity), it will be appreciated that the PS in both plasma membrane leaflets is exposed to the extracellular milieu. A similar situation exists in cell death by necrosis, where the loss of membrane integrity is either the initiating factor or occurs early in the necrotic cell death process.

The class of proteins known as annexins bind specifically and with a high affinity to PS. The structural features common to the annexines are presumably the basis for their similar Ca²⁺ and phospholipid-binding properties. Annexins are a class of compounds characterized by the ability to bind with high affinity to membrane lipids in the presence of millimolar concentrations of calcium. Annexins contain 4 or 8 repeats of a 61 amino acid domain that folds into 5 a helices. Annexins have been shown to exhibit anti-coagulatory effects that are mediated by the binding of annexins to negatively charged surface phospholipids (e.g., on activated platelets). This annexin-phospholipid binding is believed to block the activation of clotting factors by such negatively charged surface phospholipids. Annexin-V is a prototypical molecule used in the description of the present invention. The term annexin comprises native annexin purified from natural sources, such as human placenta, and annexin molecules containing a native sequence produced through e.g. genetic engineering, recombinant or other means. The term annexin further comprises modified annexins as defined below, derived from or produced by any source.

The annexin family of proteins is useful in the practice of the present invention. Annexin-V is typically found in high levels in the cytoplasm of a number of cells including the placenta, lymphocytes, monocytes, biliary and renal (cortical) tubular epithelium. Although the physiological function of annexins has not been fully elucidated, several properties of annexins make them useful as diagnostic and/or therapeutic agents. In particular, it has been discovered that annexins possess a very high affinity for anionic phospholipid surfaces, such as a membrane leaflet having an exposed surface of phosphatidylserine (PS).

The present invention is preferably practiced using annexin-V. Annexin-V is one of the most abundant annexins, it is easy to produce from natural or recombinant sources and it has a high affinity for phospholipid membranes. Human annexin-V has a molecular weight of 36 kd and high affinity (kd=7 nmol/L) for phosphatidylserine (PS). The sequence of human annexin-V can be obtained from GenBank under accession numbers U05760-U05770.

The non-radionuclide agents of the present invention are capable of recognizing a cell death state of cells. The agents allow for the initiation of early therapeutic measures by providing for the early diagnosis of a cell state that may possibly develop into a health-threatening condition.

The annexins or PSCs are conjugated with a non-radionuclide contrast agent, such as a paramagnetic contrast agent, that is detectable in an MRI, CT or optical imaging system. The conjugation of annexin-V and PSCs to a non-radionuclide contrast agent provide a huge advantage over traditional MR and CT techniques by imparting disease specific imaging while still maintaining high resolution. Examples of biocompatible paramagnetic contrast agents comprise divalent or trivalent ions of elements with an atomic number of 21 to 29, 42, 44 and 58 to 70. Suitable ions comprise chromium(III), manganese(II), iron(III), iron(II), cobalt(II), nickel(II), copper(II), praseodymium(III), neodymium(III), samarium(III) and ytterbium(III). Because of their very strong magnetic moments, gadolinium (III), terbium(III), dysoprosium(III), holmium(III), and erbium(III) are preferred. Especially preferred for the paramagnetic atom is gadolinium(III). Most preferred are gadolinium or magnetic iron containing derivatives such as superparamagnetic iron oxide.

An important feature of the present invention is that annexin-V and PSCs are not being used solely to image tumor vasculature and tumor markers, but rather any cell type undergoing cell death.

Another important feature of the present invention is the discovery and development of novel annexin-V analogues or other phosphatidylserine-binding compounds (PSC) that are less toxic than whole wild type annexin-V, and thus can be used at higher doses. An extension of this application is the identification and development of analogues or PSCs that may include peptides, small molecules, aptomers, and antibodies specific for PS or other targets specific to dead or dying cells that could be used to image PS using non-radionuclide contrast agents.

It should also be noted that the use of annexin-V in clinical settings requires a low dosage (about 1 to about 100 ug/kg). It has been observed that annexin-V begins to exert its native biological anti-coagulant activity at doses greater than about 300 ug/kg. The only conventional attempts to address this limitation have been to use annexin-V at low doses. Therefore, the development of PSCs that maintain a high affinity for PS while being biologically inactive allow higher doses to be used. For example, the genetic engineering of the one or more of the four domains (D1-D4) known to mediate calcium-dependent binding to phosphatidylserine may be employed. Since it is known that domain 1 is necessary and sufficient to mediate PS binding, it is envisioned that a peptide or other synthetic molecule that mimics the three dimensional orientation of annexin-V domain 1, such that Ca+ ions and PS head groups are oriented to allow binding, may be used in place of wild type annexin-V. Screening for high binding to PS with biochemical techniques and as well as low toxicity in animal models will allow for the selection of novel PSCs to be used for non-radionuclide imaging. Additionally, using annexin-V binding domains (D1-D4) as a “guide”, it is possible to molecularly model other compounds that contain similar PS binding properties.

In one embodiment, PSCs or homologues are created using standard molecular biology techniques such as cDNA cloning, PCR ligation, transfection, transformation, affinity purification, etc. Further, annexin-V may be linked to gadolinium (Gd) in the following manner. Typical chelates that are known to bind Gd, such as DTPA, CHXa or DOTA, are chemically coupled to the annexin-V or PSCs using standard chemistries such as isothiocyanate mediated conjugation to free amine groups.

Another important feature of this invention is the coupling of repeating units of chelated-Gd or other magnetically active contrast agents so as to provide an amplification of the signal. Although the number of annexin binding sites per cell is high (micromolar), it still may not be high enough to see a good signal against the background with Gd-based agents. To circumvent this, it is envisioned that repeating units of 10-500 Gd are linked to the annexin or PSC such that each binding event has 10-500 Gd associated with it.

An embodiment of such an amplification scheme is the creation and use of a genetically or chemically modified annexin or PSC that preferentialy contains 1-50 lysines. Each amine residue on lysine may be chemically modified to contain a chelate (DTPA or DOTA) that allows for the binding of Gd or other magnetic metals. Although lysine is the residue of choice, other repeating amino acid residues that may be chemically modified to chelate metals may be used.

Gadolinium has been studied extensively in vivo and is currently used in clinical applications. Therefore, MR image reconstruction, pulse sequences development and other imaging parameters have already been developed that may readily allow for the imaging of Gd-based agents.

In addition, iron containing nanoparticles having functionalized coated surfaces that allow annexin-V or PSCs to be attached to the particle may also be used. Iron containing nanoparticles provide an advantage over Gd by yielding a much higher signal per molecule. This is due to the high number of iron nuclei that may be packed within one nanoparticle. Iron containing MR contrast agents may also be used in vivo.

In one embodiment, the non-radionuclide contrast agent may comprise a polymer coating. The polymer may be responsive to environmental changes. The coating may comprise an ionic or a non-ionic coating. For example, the coating may comprise a homopolymer, block copolymer, or a graft comprising hydrophilic and hydrophobic blocks of ionic and non-ionic nature.

After the labeled annexin is administered, it is allowed to localize to dead and dying cells, indicating a region of possible disease. One of skill in the art will appreciate that it may be desirable to perform the imaging at times between about 5 minutes to about 48 hours, more preferably between about 10 minutes to about 4 hours, and even more preferably from about 5 minutes to about 30 minutes. In all of the above cases, a reasonable estimate of the time to achieve localization and clearance may be made by one skilled in the art.

Monitoring apoptosis at discrete time intervals using a non-radiolabeled annexin or PSCs may be used for determining the time required for a cell to die. Testing may also lead to the development of new drugs and therapies for a variety of diseases. In addition, the methods may be used to monitor the progress of treatment, monitor the progress of disease, or both. Further, they are important diagnostic tools may be used to aid in early detection of certain diseases.

To obtain an image, a patient is instructed to lie on a narrow table that slides into the center of a scanner. Depending on the study being performed, the patient may need to lie on his/her stomach, back, or side. When the contrast media is administered, an IV may be placed in a small vein of a hand or arm. Much like standard photographic cameras, subject motion causes blurred images in CT. Therefore, the technologist operating the scanner and supervising the patient gives instructions through an intercom when the patient should hold his/her breath and not move.

As an exam takes place, the patient advances at small intervals through the scanner. Modern “spiral” scanners may perform the examination in one continuous motion of the table. Generally, complete scans take only a few minutes, however, additional contrast-enhanced or high-resolution scans add to the scan time. The newest multi-detector scanners image the entire body, head-to-toe, in less than 30 seconds.

In one example, a patient may be asked to drink the oral contrast either immediately prior to or 4 to 6 hours before a CT scan. The health care provider may also advise fasting (no solids or liquids) for 4 to 6 hours before the scan. The labeled annexin of the present invention may be applied to cells or may be administered to a subject intraarterially or intravenously and allowed to circulate in the bloodstream. The labeled annexin may be used normally in the form of a suspension or solution in a solvent, such as distilled water for injection, physiological saline and the like. In specific applications, a pharmacologically acceptable additive may be used, such as a carrier, expedient or the like. Preferably, the labeled annexin is administered in the form of an aqueous agent emulsion or suspension. The additive used may vary depending on the mode of administration, administration route, and the like. Examples of additives for intravenous injections comprise buffers, antibacterial agents, stabilizers, solubilizers, and excipients that are used alone or in combination. Examples of optical dyes that may coupled to the annexin or PSCs are cyanine based optical dyes, such as Cy3, Cy5 and other indocyanine based dyes.

An imaged region may comprise the entire patient or only a portion of the patient that needs to be diagnosed or monitored for cell death. For example, the region may comprise an appendage, a part of an appendage, the head, the central nervous system, and an internal cavity such as the thoracic or peritoneal cavity. In specific embodiments, the region may comprise only a selected organ or portion thereof. For example, the method may be applied to an analysis of cell death in the central nervous system, kidney, brain, heart, liver, spleen, lungs, bone marrow, a portion of any of the above, etc. Further, the region analyzed may be restricted to a tumor, e.g., in a cancer patient undergoing treatment designed to cause cell death in the tumor, such as chemotherapy.

It may be desired to record the effects of a treatment on the distribution and/or localization of cell death over time. The imaging may be repeated at selected time intervals in order to construct a series of images. The interval may comprise seconds, minutes, days, weeks, months, and years.

The foregoing is a description of a preferred embodiment of the invention which is given here by way of example only. The invention is not to be taken as limited to any of the specific features as described, but comprehends all such variations thereof as come within the scope of the appended claims.

What is claimed is:

1. A method for imaging cell death in a subject in vivo, comprising:
labeling a phosphatidylserine (PS) binding compound (PSC) with a non-radionuclide contrast agent;
administering to the subject the non-radionuclide contrast agent labeled PSC;
allowing the labeled PSC to specifically bind to the surface of dead and dying cells; and
imaging the subject for the purpose of detecting the dead and dying cells.

2. The method of claim 1, wherein the non-radionuclide contrast agent comprises at least one of the following: gadolinium (III), terbium(III), dysoprosium(III), holmium(III), erbium(III), chromium(III), manganese(II), iron(III), iron(II), cobalt(II), nickel(II), copper(II), praseodymium(III), neodymium(III), samarium(III) and ytterbium(III).

3. The method of claim 1, wherein the non-radionuclide contrast agent comprises at least one of the following: a magnetic resonance contrast agent, a computed tomography contrast agent, and an optical contrast agent.

4. The method of claim 1, wherein the labeled PSC has a high affinity for binding with phosphatidylserine (PS).

5. The method of claim 1, wherein imaging the subject comprises imaging by one of the following: magnetic resonance imaging, computed tomography, and optical imaging.

6. The method of claim 1, wherein the dead and dying cells comprise cells that have undergone at least one of the following: apoptosis, necrosis, lysis, and senescence.

7. The method of claim 1, wherein the dead and dying cells translocate phosphatidylserine from an inner cell membrane to an outer extracellular surface of the cells.

8. The method of claim 1, wherein the PSC comprises an annexin.

9. The method of claim 1, wherein the annexin comprises annexin-V.

10. The method of claim 9, wherein the annexin comprises a derivative of annexin-V having a high affinity for phosphatidylserine.

11. The method of claim 1, wherein the PSC is conjugated to a chelate via an isothyiocyanate-amine linkage and the chelate is used to bind gadolinium.

12. The method of claim 2, wherein repeating units of chelated-Gd are used to provide an amplification of a signal.

13. The method of claim 12, wherein repeating units of about 10 to about 500 gadolinium are linked to the annexin or PSC such that each binding event has about 10 to about 500 Gd associated with it.

14. The method of claim 8, wherein the annexin or PSC contains about 1 to about 50 lysines.

15. The method of claim 9, wherein the annexin-V contains about 1 to about 50 lysines.

16. The method of claim 15, wherein the lysines are conjugated to a chelate via an isothiocyanate linkage.

17. The method of claim 1, wherein the amount of labeled PSC administered is from about 0.001 to about 20 mg protein/kg.

18. The method of claim 1, wherein the amount of labeled PSC administered is from about 1 to about 1000 μg protein/kg.

19. The method of claim 1, further comprising determining the extent of binding of the labeled PSC to the phosphatidylserine on a phospholipid surface.

20. A conjugate comprising:
a phosphatidylserine (PS) binding compound (PSC); and
a non-radionuclide contrast agent.

21. The conjugate of claim 20, wherein the non-radionuclide contrast agent comprises at least one of the following: a magnetic resonance contrast agent, a computed tomography contrast agent, and an optical contrast agent.

22. The conjugate of claim 20, wherein the non-radionuclide contrast agent comprises at least one of the following: gadolinium (III), terbium(III), dysoprosium(III), holmium(III), erbium(III), chromium(III), manganese(II), iron(III,) iron(II), cobalt(II), nickel(II), copper(II), praseodymium(III), neodymium(III), samarium(III) and ytterbium(III).

23. The conjugate of claim 20, wherein the PSC comprises an annexin analogue specific for phosphatidylserine comprising at least one of the following: peptides, small molecules, aptomers, and antibodies.

24. The conjugate of claim 20, wherein the PSC is conjugated to a chelate via an isothyiocyanate-amine linkage and the chelate is used to bind gadolinium.

25. The conjugate of claim 20, wherein the PSC comprises an annexin.

26. The conjugate of claim 25, wherein the annexin comprises annexin-V.

27. The conjugate of claim 20, wherein the PSC specifically binds with phosphatidylserine (PS).

28. The conjugate of claim 20, wherein the conjugate is used for imaging dead and dying cells.

29. The conjugate of claim 28, wherein the dead and dying cells have undergone at least one of the following: apoptosis, necrosis, lysis, and senescence.

30. The conjugate of claim 28, wherein the imaging comprises at least one of the following: magnetic resonance imaging, computed tomography, and optical imaging.

31. The conjugate of claim 28, wherein the dead and dying cells have translocated phosphatidylserine from an inner membrane of the cells to an outer extracellular surface of the cells.

32. The conjugate of claim 20, wherein repeating units of chelated-Gd are used to provide an amplification of a signal.

33. The conjugate of claim 32, wherein repeating units of about 10 to about 500 gadolinium are linked to the annexin or PSC such that each binding event has about 10 to about 500 Gd associated with it.

34. The conjugate of claim 25, wherein the annexin or PSC contains about 1 to about 50 lysines.
